(19) United States
(12) Patent Application Publication
(10) Pub. No.: US 2026/0279563 A1
(43) Pub. Date: Sep. 17, 2026

(54) AUTOMATED REMOTE SUPPORT REQUESTS BASED ON CONSOLE EXTRACTION OF SYSTEM WARNINGS IN THE CONTEXT OF A RUNNING IMAGING EXAMINATION

(71) Applicant: KONINKLIJKE PHILIPS N.V., EINDHOVEN (NL)

(72) Inventors: THOMAS ERIK AMTHOR, HAMBURG (DE); TANJA NORDHOFF, HAMBURG (DE); SASCHA KRUEGER, HAMBURG (DE); JULIEN THOMAS SENEGAS, HAMBURG (DE)

(21) Appl. No.: 19/167,668

(22) PCT Filed: Mar. 18, 2024
(86) PCT No.: PCT/EP2024/057111

§ 371 Date: Sep. 22, 2025

Related U.S. Application Data
(60) Provisional application No. 63/455,630, filed on Mar. 30, 2023.

(51) Int. Cl. G16H 40/67; G01R 33/54; G01R 33/58; G06F 3/14; G06V 30/10; G16H 40/20; G16H 40/40

(52) U.S. Cl. CPC G16H 40/67; G01R 33/546; G01R 33/58; G16H 40/20; G16H 40/40; G06F 3/1454; G06V 30/10; G06V 2201/02

(57) ABSTRACT

A non-transitory computer readable medium (26s) stores instructions executable by at least one electronic processor (14s) to perform a method (100) for assisting an imaging examination of a patient. The method includes receiving examination monitoring information (17, 18, 27) related to the imaging examination and transmitting the examination monitoring information (17, 18, 27) to a remote expert (RE), the examination monitoring information including at least a screen mirroring data stream (27) of a controller (10) of an imaging device (2) used to perform the imaging examination of the patient; tracking a status of the imaging examination from the received examination monitoring information; determining one or more indicators of an issue with the imaging examination based on the tracked status of the imaging examination; and outputting a warning indicative of the determined one or more indicators of the issue with the imaging examination.

The following relates generally to the imaging arts, remote imaging assistance arts, remote imaging examination monitoring arts, and related arts.

### BACKGROUND

Medical imaging, such as computed tomography (CT) imaging, magnetic resonance imaging (MRI), positron emission tomography (PET) imaging, fluoroscopy imaging, and so forth, is a valuable component of providing medical care, and is used in a wide range of medical fields, such as cardiology, oncology, neurology, orthopedics, to name a few. The operator of the medical imaging device used to acquire the medical images is typically a trained technologist, while interpretation of the medical images is often handled by a medical specialist such as a radiologist.

CT and MRI are powerful imaging modalities used by clinicians and researchers to non-invasively visualize human anatomy and pathology in great detail. Image contrast depends primarily on the parameters and sequences used to acquire the images, and CT and MRI exams are categorized by protocol type depending on which organ/body part the scan is being ordered for. CT protocols vary in scan parameters and MRI protocols typically include numerous distinct sequences to obtain different anatomic and pathologic information from images.

Remote and distributed working environments are gaining importance in all areas of industry and even in Healthcare. A Radiology Operations Command Center (ROCC) system can be a virtualization solution for radiology. This system includes capturing and transmission of the local console screen to a remote command center, optionally along with other types of examination information capture such as a bay video camera capturing activity in the imaging bay. The ROCC thus provides situational awareness to an expert at the remote command center, who can advise or guide the local imaging technologist performing the imaging examination via a video call or the like. The ROCC beneficially enables a single expert to assist in a number of ongoing imaging examinations, which may be widely distributed geographically.

Many disruptions in imaging workflow, in particular in MR imaging, occur because a system state or warning message has been disregarded or misinterpreted, resulting in safety risk or bad image quality and the need for a scan repetition. Some disruption examples can include a missing or bad quality electrocardiogram (ECG) trigger signal in cardiac examinations, low signal-to-noise (SNR) coil signals in general, in-exam spurious test results (i.e., a leak in a radiofrequency (RF) cage or not properly closed door), low battery states of accessories, inappropriate selected motion compensation/fat suppression strategy for given patient signals, high level of specific absorption rate or peripheral nerve simulation, and so forth.

The implication of system state or warning messages for a specific activity may not always be apparent to all staff, especially when staff members are inexperienced. Some signals may only affect the workflow if they occur in a specific combination. In some cases, expert support from the ROCC would be beneficial to solve the issue, but the local operator may not even be aware of this need.

The following discloses certain improvements to overcome these problems and others.

### SUMMARY

In one aspect, a non-transitory computer readable medium stores instructions executable by at least one electronic processor to perform a method for assisting an imaging examination of a patient. The method includes receiving examination monitoring information related to the imaging examination and transmitting the examination monitoring information to a remote user, the examination monitoring information including at least a screen mirroring data stream of a controller of an imaging device used to perform the imaging examination of the patient; tracking a status of the imaging examination from the received examination monitoring information; determining one or more indicators of an issue with the imaging examination based on the tracked status of the imaging examination; and outputting a warning indicative of the determined one or more indicators of the issue with the imaging examination.

In another aspect, an apparatus is disclosed for assisting an imaging examination of a patient performed using an imaging device controlled by an imaging device controller. The apparatus comprises: a screen mirroring connection configured to perform screen mirroring of a display of the imaging device controller; and at least one electronic processor programmed to perform a method for assisting an imaging examination of a patient. The method includes: receiving examination monitoring information related to the imaging examination and transmitting the examination monitoring information to a remote user, the examination monitoring information including at least a screen mirroring data stream of the display of the imaging device controller acquired by the screen mirroring connection; tracking a status of the imaging examination from the received examination monitoring information; determining one or more indicators of an issue with the imaging examination based on the tracked status of the imaging examination; and outputting, on a display device, a warning indicative of the determined one or more indicators of the issue with the imaging examination.

In another aspect, an imaging system is disclosed, including: an imaging device; an imaging device controller operatively connected to control the imaging device; a camera arranged to acquire video of at least an area of the imaging device; and an apparatus as set forth in the immediately preceding paragraph for assisting an imaging examination of a patient performed using the imaging device controlled by the imaging device controller. In some embodiments, the examination monitoring information further includes video acquired by the camera.

In another aspect, a method for assisting an imaging examination of a patient includes receiving examination monitoring information related to the imaging examination and transmitting the examination monitoring information to a remote user, the examination monitoring information including at least a screen mirroring data stream of a controller of an imaging device used to perform the imaging examination of the patient; tracking a status of the imaging examination from the received examination monitoring information; determining one or more indicators of an issue with the imaging examination based on the tracked status of the imaging examination; and outputting a warning indicative of the determine one or more indicators of the issue with the imaging examination.

One advantage resides in reducing imaging workflow interruptions.

Another advantage resides in analyzing system state and warning messages on imaging console screens to generate automated support requests.

Another advantage resides in evaluating system state and warning messages on imaging console screens based on an image quality and workflow of an imaging examination.

Another advantage resides in alerting a technologist performing an imaging examination of a potential workflow interruption in the examination.

Another advantage resides in assisting a remote expert who may be monitoring multiple imaging examinations by providing automated support requests when the local operator may be unaware of the problem and the remote expert may have difficulty discerning the problem via the remote feeds the expert is monitoring.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically shows an illustrative apparatus for providing remote assistance in accordance with the present disclosure.

FIG. 2 shows an example flow chart of operations suitably performed by the apparatus of FIG. 1.

FIG. 3 shows a schematic of a server of the apparatus of FIG. 1.

FIGS. 4A and 4B show other embodiments of the server of FIG. 3.

### DETAILED DESCRIPTION

This application relates to a Radiology Operations Command Center (ROCC), which provides the local operator of a medical imaging device with telephonic or video call access to a remote expert who can provide assistance during a medical imaging examination. The ROCC infrastructure includes a bay camera that provides the remote expert with video of the imaging bay, a physical or software-based screen capture/duplication to present a real-time copy of the controller display to the remote expert, and optionally other data sources such as a bay microphone. Thus, the remote expert has real-time awareness of the status of the imaging examination.

In the ROCC setup, a single remote expert may be assigned to provide assistance as needed to multiple imaging bays. Put another way, the remote expert may be simultaneously overseeing multiple imaging examinations. This is feasible because most of the time the local operators will not need assistance from the remote expert, and usually the local operator will contact the remote expert when assistance is needed. While this beneficially enables providing ROCC services to many imaging bays at the same or different hospitals or radiology laboratories, it does present a challenge for the remote expert. In particular, if the local operator does not recognize a problem when it arises, then the remote expert may also fail to recognize the problem due to overloading with overseeing multiple examinations or simply because the remote situation of the remote expert makes it more difficult to recognize certain types of problems.

The following discloses an improvement in which the ROCC infrastructure itself monitors the imaging examination to detect certain types of problems and automatically alert the remote expert that assistance may be needed.

Such detection relies in part on examination context gathered by the ROCC. Since the ROCC infrastructure captures the controller display and bay camera video in real time, it can track the status of the imaging examination including relevant information such as the imaging modality (known a priori), the imaging scan being performed (extracted, for example, by OCR analysis of text on the controller display or matching graphical user interface (GUI) dialogs with templates), the location of the patient (being loaded into the imaging bore, or in the bore, or being removed from the bore), and/or so forth. From the patient electronic medical record (EMR) or other sources, the ROCC can identify patient-specific considerations impacting the imaging examination, such as an overweight patient or a relevant chronic medical condition of the patient.

In some embodiments, the ROCC maintains a database of local operator profiles, including such information as their amount of experience (optionally subdivided by imaging modality, imaging device vendor, et cetera), educational level, and so forth.

In some embodiments, the ROCC can analyze the captured controller display content to automatically identify warnings, error messages, or other indicators of possible problems. In view of the gathered examination context and the local operator profile, a determination is made as to whether a given warning, error message or the like (or combination of such indicators) should trigger an alert to the remote expert, and if so, the alert is issued.

As an example, some imaging examinations rely on cardiac gating using an electrocardiogram (ECG) signal, or respiratory gating using a respiratory monitor signal, to acquire data at specific cardiac or respiratory phases. When a gated imaging sequence is performed, the ECG or respiratory monitor must be connected to provide the gating signal. When an ungated imaging sequence is performed, the medical imaging device itself may supply a default gating signal (e.g. a pulse every second). If the ROCC detects a warning that a gating signal is not being supplied, it can then determine based on the type of imaging examination (gated or ungated) whether this missing gating signal is actually a problem, and issue an alert to the remote expert only on the combination of a missing gating signal and a gated imaging sequence being performed.

As another example, if the ROCC detects that a certain type of local MR coil is not being used in an MRI imaging sequence where such a local MR coil is required, this information can trigger an alert to the remote expert. As yet another example related to MRI, an open door or a leak in the RF room Faraday cage can result in image quality degradation. The ROCC can perform image analysis to detect this type of image degradation, and if detected, an alert can be issued to the remote expert.

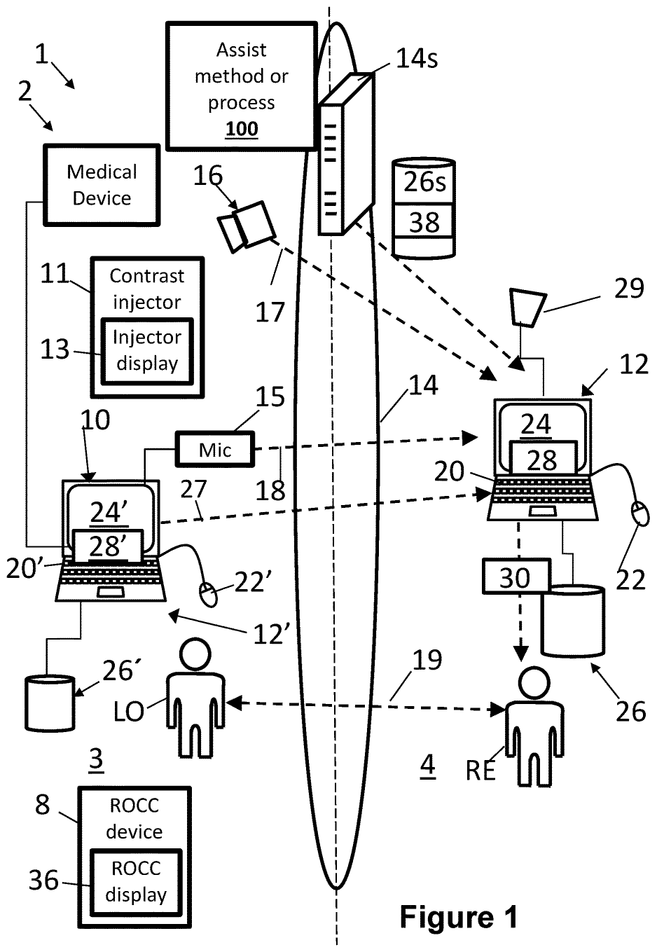

With reference to FIG. 1, an apparatus 1 for providing assistance from a remote medical imaging expert RE (shortened to “remote expert” hereinafter) to a local operator LO (e.g., an imaging technologist) is shown. As shown in FIG. 1, the local operator LO, who operates an imaging device (also referred to as an image acquisition device, imaging device, and so forth) 2, is located in a medical imaging device bay 3, and the remote expert RE is disposed in a remote service location or center 4. It should be noted that the “remote expert” RE may not necessarily directly operate the imaging device 2, but rather provides assistance to the local operator LO in the form of advice, guidance, instructions, or the like. The remote location 4 can be a remote service center, a radiologist's office, a radiology department, and so forth. The remote location 4 may be in the same building as the imaging device bay 3 (this may, for example, in the case of a remote user (e.g., remote expert) RE who is a radiologist tasked with peri-examination image review), but more typically the remote location 4 and the imaging device bay 3 are in different buildings, and indeed may be located in different cities, different countries, and/or different continents. In general, the remote location 4 is remote from the imaging device bay 3 in the sense that the remote expert RE cannot directly visually observe the imaging device 2 in the imaging device bay 3 (hence optionally providing a video feed as described further herein).

The image acquisition device 2 can be a Magnetic Resonance (MR) imaging device, a Computed Tomography (CT) imaging device; a positron emission tomography (PET) imaging device; a single photon emission computed tomography (SPECT) imaging device; an X-ray imaging device; an ultrasound (US) imaging device; or an imaging device of another modality. The imaging device 2 may also be a hybrid imaging device such as a PET/CT or SPECT/CT imaging system. While a single image acquisition device 2 is shown by way of illustration in FIG. 1, more typically a medical imaging laboratory will have multiple image acquisition devices, which may be of the same and/or different imaging modalities. For example, if a hospital performs many CT imaging examinations and relatively fewer MRI examinations and still fewer PET examinations, then the hospital's imaging laboratory (sometimes called the “radiology lab” or some other similar nomenclature) may have three CT scanners, two MRI scanners, and only a single PET scanner. This is merely an example. Moreover, the remote location 4 may provide service to multiple hospitals. The local operator LO controls the imaging device 2 via an imaging device controller 10. The remote expert or other user RE is stationed at and uses a remote electronic processing device 12 (or, more generally, an electronic controller 12).

Some types of imaging modalities and some types of imaging examinations may employ a contrast agent. For example, some types of MRI angiography imaging examinations employ a gadolinium-based magnetic contrast agent to observe blood flow into and out of an anatomical organ or region. To provide for such contrast-enhanced imaging, a programmable contrast injector 11 with a display 13 is configured to inject the patient with a contrast agent. In such an embodiment, the injector display 13 may be monitored by a further ROCC sensor such as a camera viewing the injector display 13.

As used herein, the term “imaging device bay” (and variants thereof) refer to a room containing the imaging device 2 and also any adjacent control room containing the imaging device controller 10 for controlling the imaging device. For example, in reference to an MRI device, the imaging device bay 3 can include a radiofrequency (RF) shielded room containing the MRI device 2, as well as an adjacent control room housing the imaging device controller 10, as understood in the art of MRI devices and procedures. On the other hand, for other imaging modalities such as CT, the imaging device controller 10 may be located in the same room as the imaging device 2, so that there is no adjacent control room and the medical bay 3 is only the room containing the imaging device 2. In addition, while FIG. 1 shows a single imaging device bay 3, it will be appreciated that the remote location 4 (and more particularly the remote electronic processing device 12) is in communication with multiple medical bays via a communication link 14, which typically comprises the Internet augmented by local area networks at the remote expert RE and local operator LO ends for electronic data communications. In addition, while FIG. 1 shows a single remote location 4, it will be appreciated that the imaging device bay 3 is in communication with multiple medical bays via the communication link 14.

As diagrammatically shown in FIG. 1, in some embodiments, a camera 16 (e.g., a video camera) is arranged to acquire a video stream or feed 17 of a portion of a workspace of the imaging device bay 3 that includes at least the area of the imaging device 2 where the local operator LO interacts with the patient, and optionally may further include the imaging device controller 10. In other embodiments, a microphone 15 is arranged to acquire an audio stream or feed 18 of the workspace that includes audio noises occurring within the imaging device bay 3 (e.g., verbal instructions by the local operator LO, questions from the patient, and so forth). The video stream 17 and/or the audio stream 18 is sent to the remote electronic processing device 12 via the communication link 14, e.g., as a streaming video feed received via a secure Internet link.

The communication link 14 also provides a natural language communication pathway 19 for verbal and/or textual communication between the local operator LO and the remote expert or other user RE. For example, the natural language communication pathway 19 may be a Voice-Over-Internet-Protocol (VOIP) telephonic connection, an online video chat link, a computerized instant messaging service, or so forth. Alternatively, the natural language communication pathway 19 may be provided by a dedicated communication link that is separate from the communication link 14 providing the data communications 17, 18, e.g., the natural language communication pathway 19 may be provided via a landline telephone. In some embodiments, the natural language communication pathway 19 allows a local operator LO to call a selected remote expert RE. The call, as used herein, can refer to an audio call (e.g., a telephone call), a video call (e.g., a Skype or Facetime or other screen-sharing program), or an audio-video call. In another example, the natural language communication pathway 19 may be provided via a ROCC device 8 with a display device 36. For example, an “app” can run on the ROCC device 8 (operable by the local operator LO) and the remote electronic processing device 12 (operable by the remote expert RE) to allow communication (e.g., audio chats, video chats, and so forth) between the local operator and the remote expert. The ROCC Device 8 is for example a tablet, mobile phone, desktop PC, workstation, etc.

FIG. 1 also shows the remote location 4 which includes the remote electronic processing device 12, such as a workstation, a workstation computer, or more generally a computer, which is operatively connected to receive and present the video feed 17 of the imaging device bay 3 from the camera 16 and/or to the audio feed 18. Additionally or alternatively, the remote workstation 12 can be embodied as a server computer or a plurality of server computers, e.g., interconnected to form a server cluster, cloud computing resource, or so forth. The workstation 12 includes typical components, such as an electronic processor 20 (e.g., a microprocessor), at least one user input device (e.g., a mouse, a keyboard, a trackball, and/or the like) 22, and at least one display device 24 (e.g., an LCD display, plasma display, cathode ray tube display, and/or so forth). In some embodiments, the display device 24 can be a separate component from the remote electronic processing device 12. The display device 24 may also comprise two or more display devices. The electronic processor 20 is operatively connected with a one or more non-transitory storage media 26. The non-transitory storage media 26 may, by way of non-limiting illustrative example, include one or more of a magnetic disk, RAID, or other magnetic storage medium; a solid state drive, flash drive, electronically erasable read-only memory (EEROM) or other electronic memory; an optical disk or other optical storage; various combinations thereof; or so forth; and may be for example a network storage, an internal hard drive of the workstation 12, various combinations thereof, or so forth. It is to be understood that any reference to a non-transitory medium or media 26 herein is to be broadly construed as encompassing a single medium or multiple media of the same or different types. Likewise, the electronic processor 20 may be embodied as a single electronic processor or as two or more electronic processors. The non-transitory storage media 26 stores instructions executable by the at least one electronic processor 20. The instructions include instructions to generate a graphical user interface (GUI) 28 for display on the remote display device 24. The video feed 17 from the camera 16 can also be displayed on the display device 24, and the audio feed 18 can be output on the remote electronic processing device 12 via a loudspeaker 29. In some examples, the audio feed 18 can be an audio component of an audio/video feed (such as, for example, outputting the audio feed 18 as a video cassette recorder (VCR) device would operate).

FIG. 1 shows an illustrative local operator LO, and an illustrative remote expert RE (e.g., a senior imaging technologist, radiologist or the like). However, in a Radiology Operations Command Center (ROCC) as contemplated herein, the ROCC provides a staff of remote experts (e.g., senior imaging technologists, radiologists, or so forth) who are available to assist local operators LO at different hospitals, radiology labs, or the like. Each remote expert RE can operate a corresponding remote electronic processing device 12. The ROCC may be housed in a single physical location or may be geographically distributed. For example, in one contemplated implementation, the remote expert RE are recruited from across the United States and/or internationally in order to provide a staff of remote experts with a wide range of expertise in various imaging modalities and in various imaging procedures targeting various imaged anatomies. A server computer 14s can be in communication with the imaging bay 3 and the remote location 4 with one or more non-transitory storage media 26s. The non-transitory storage media 26s may, by way of non-limiting illustrative example, include one or more of a magnetic disk, RAID, or other magnetic storage medium; a solid state drive, flash drive, electronically erasable read-only memory (EEROM) or other electronic memory; an optical disk or other optical storage; various combinations thereof; or so forth; and may be for example a network storage, an internal hard drive of the server computer 14s, various combinations thereof, or so forth. It is to be understood that any reference to a non-transitory medium or media 26s herein is to be broadly construed as encompassing a single medium or multiple media of the same or different types. Likewise, the server computer 14s may be embodied as a single electronic processor or as two or more electronic processors. The non-transitory storage media 26s stores instructions executable by the server computer 14s.

The imaging device controller 10 in the imaging device bay 3 also includes similar components as the remote electronic processing device 12 disposed in the remote location 4. Except as otherwise indicated herein, features of the imaging device controller 10, which includes a local workstation 12′, disposed in the imaging device bay 3 similar to those of the remote workstation 12 disposed in the remote location 4 have a common reference number followed by a “prime” symbol, and the description of the components of the imaging device controller 10 will not be repeated. In particular, the imaging device controller 10 is configured to display a GUI 28′ on a display device or controller display 24′ that presents information pertaining to the control of the imaging device 2, such as configuration displays for adjusting configuration settings an alert 30 perceptible at the remote location when the status information on the imaging examination satisfies an alert criterion of the imaging device 2, imaging acquisition monitoring information, presentation of acquired medical images, and so forth. A screen mirroring data stream 27 carries the content presented on the display device 24′ of the imaging device controller 10. The screen mirroring data stream 27 is produced by a screen mirroring connection configured to perform the screen mirroring of the display 24′ of the imaging device controller 10. The screen mirroring data stream 27 can be acquired in various ways. In one approach, the screen mirroring data stream 27 can be acquired by a screen mirroring connection comprising screen mirroring software running on the imaging device controller 10. In another approach, the screen mirroring connection comprises screen scraping hardware interposed between the display device 24′ of the imaging device controller 10 and the at least one electronic processor 20′ (e.g., video card or the like) that outputs the video signal. In this latter approach, the screen scraping hardware may, by way of nonlimiting illustrative example, include a video splitter that splits the video signal, with one video signal being sent to the display device 24′ and the other being sent to the remote electronic processing device 12 as the screen mirroring data stream 27. The communication link 14 allows for screen sharing between the display device 24 in the remote location 4 and the display device 24′ in the imaging device bay 3. The GUI 28′ includes one or more dialog screens, including, for example, an examination/scan selection dialog screen, a scan settings dialog screen, an acquisition monitoring dialog screen, among others. The GUI 28′ can be included in the video feed 17 and displayed on the remote workstation display 24 at the remote location 4.

The server 14s performs a method or process 100 for assisting a medical imaging examination of a patient performed using the imaging device 2 (i.e., by assisting a local operator LO of the imaging device 2 during imaging examinations by a remote expert RE). The method or process 100 is further operative to provide assistance between local operators LO, as disclosed herein. In embodiments disclosed herein, the method 100 includes tracking a status of the imaging examination from the examination monitoring information, determining indicator(s) of an issue with the imaging examination based on the tracked status of the imaging examination, and outputting a warning indicative of the determined one or more indicators of the issue with the imaging examination to the local operator LO and/or the remote expert RE. This aspect can advantageously enable automated detection and notification of problems that the local operator LO may be unaware of, and that the remote expert RE may have difficulty recognizing given limitations in the situational awareness provided to the remote expert by the various feeds, and the workload on the remote expert RE who may be monitoring multiple ongoing imaging examinations simultaneously.

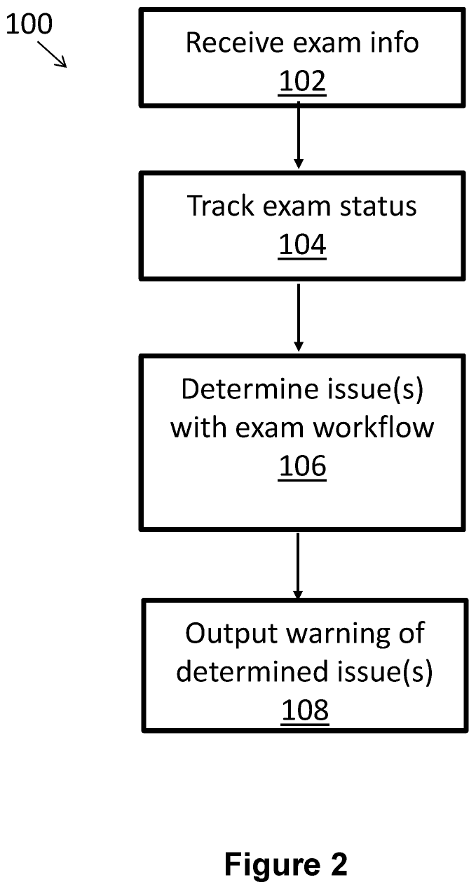

With reference to FIG. 2, and with continuing reference to FIG. 1, an illustrative embodiment of the method 100, and specifically the aspect of automated detection and notification of issues with the imaging examination, is diagrammatically shown as a flowchart. To begin the method 100, at an operation 102, during an imaging procedure, information related to a current imaging examination of the patient is received at the server computer 14s and transmitted to the remote electronic processing device 12. The received information can include a variety of different types of information. For example, the received information can include information obtained by analyzing the video stream 17, audio stream 18, and/or the screen sharing video stream 27 providing a copy of the content displayed on the display device 24′ of the imaging device controller 10. The received information can also include, for example, inputs related to the medical examination that are received from the remote monitoring by the ROCC and/or input by the local operator LO (i.e., the local imaging technologist LO) via the ROCC device 8, information from prior studies or imaging examinations, reimbursement guidelines for imaging examinations; inputs related to the imaging examination that are input by a radiologist (for example, as annotations to the examination order made by the radiologist) and/or the remote expert RE via the remote electronic processing device 12; and/or so forth. The received information is then transmitted to the remote electronic processing device 12 for analysis by the remote expert RE. The examination monitoring information 17, 18, 27 can also include one or more of a modality of the imaging device 2, an imaging scan being performed and extracted by optical character recognition (OCR) analysis of text on the controller display or matching graphical user interface (GUI) dialogs with templates, a location of the patient relative to the imaging device 2, and so forth.

At an operation 104, a status of the imaging examination is tracked from the received examination monitoring information. The tracking operation 104 can be performed, for example, by performing an OCR process on text from the examination monitoring information 17, 18, 27, or matching one or more templates 38 stored in the server computer 14s with an image of the examination monitoring information 17, 18, 27. As used herein, The status of the imaging examination that may be tracked based on the received examination monitoring information may include numerous components such as examination and/or system states and/or warning messages, possibly including time sequence aspects. For example, components of the status of the imaging examination may include error messages displayed on the controller display 24″, the presence or absence of input signals to the imaging controller 10 (e.g., in the case of MRI presence or absence of a signal indicating a local MR coil is operatively connected with the MRI scanner), active or queued imaging programs or recipes, the current settings of such programs or recipes (e.g., TR, TE, and other settings in the case of MRI), and so forth. Some components of the status may be directly determined from the received examination monitoring information 17, 18, 27, such as error messages displayed on the controller display 24′. Other components of the status may be derived from the received examination monitoring information 17, 18, 27, possibly in conjunction with other available information. For example, during a CT examination, the specific absorption rate (SAR) may be a status component that is derived from the X-ray beam settings and patient characteristics such as patient weight. Some components of the status may be derived from time-related aspects of the received examination monitoring information 17, 18, 27. For example, in the case of an imaging examination that measures inflow and washout of a contrast agent, the sequence of events may be critical, for example including a reference image acquisition followed by triggering of the contrast injector 11 to intravascularly inject a contrast bolus followed by a contrast-enhanced imaging sequence designed to acquire a time-sequence of images encompassing the expected time of contrast agent inflow into the anatomical region of interest and subsequent washout of the contrast agent from the anatomical region. In such a case, the status of the imaging examination may include the presence or absence of appropriate setup of the contrast injector and suitable programming of the imaging controller 10 with the reference and contrast-enhanced imaging sequences with suitable parameters (e.g., acquisition time intervals) for these sequences. Also, if the expected contrast imaging workflow includes the imaging controller automatically triggering the contrast injector 11, then the status may include whether a signal indicating proper connection of the contrast injector 11 with the imaging controller is present or absent. These are merely nonlimiting illustrative examples of some suitable components of the status of the imaging examination that may be tracked based on the received examination monitoring information.

At an operation 106, one or more indicators of an issue with the imaging examination are determined based on the tracked status of the imaging examination. This can be performed in a variety of manners. In one example, an error message displayed on the controller display 24″, where the indicator of the issue is the error message and/or the issue being identified by the error message. In another example the issue is determined as a deviation of the tracked status of the imaging examination from an expected imaging examination workflow that is expected for the imaging examination. In this example, the expected workflow is obtained based on the received examination monitoring information 17, 18, 27.

In some embodiments, the issue determination operation 106 includes determining an absence or a poor quality of an operative connection with the imaging device 2 of a component that is expected to be operatively connected based on an expected imaging examination workflow that is expected for the imaging examination. In one example, the component is an electrocardiogram (ECG) or respiratory monitor and the operative connection with the ECG or respiratory monitor comprises a gating signal expected to be input to the imaging device 2 by the ECG or respiratory monitor. In another example, the imaging device 2 comprises a magnetic resonance imaging (MRI) scanner, the component is an MRI coil, and the operative connection comprises a wired or wireless communication connection between the MRI coil and the MRI scanner 2. In another example, the imaging device 2 comprises an MRI scanner, and the issue is determined as a radio frequency (RF) leakage of a Faraday cage enclosing the MRI scanner (i.e., from a door being left open) based on analysis of an MRI image acquired by the MRI scanner 2 during the imaging examination.

In other embodiments, the issue determination operation 106 includes identifying, from patient data, patient-specific considerations impacting the imaging examination (e.g., weight, gender, presence of implants, and so forth). In another example, the issue determination operation 106 includes identifying, from medical facility personnel data, a local operator profile for an operator performing the imaging examination (e.g., a role, a credential level, and/or an expertise level of the local operator LO). The issue is determined based on the tracked status of the imaging examination and further based on the local operator profile of the operator.

These are all merely examples of the issue determination operation 106, and should not be construed as limiting.

At an operation 108, a warning indicative of the determined one or more indicators of the issue with the imaging examination is output. For example, the warning can be output on the display device 36 of the ROCC device 8 operable by the local operator LO and/or the display device 24 of the remote electronic processing device 12 operable by the remote expert RE. In some examples, the local operator LO can provide an input that is received by the display device 36 of an indication that the warning is resolved (the remote expert RE can provide a similar input on the remote electronic processing device 12). In some examples, the server computer 14s is programmed to determine whether the one or more determined indicators of the issue with the imaging examination satisfies a predetermined warning threshold, and output the warning when the one or more determined indicators of the issue exceed the predetermined warning threshold.

In some embodiments, the method 100 can further include establishing the natural communication pathway 19 between the local operator LO and the remote expert RE based on the customized imaging protocol 142. This may occur in response to the local operator LO making a request for remote expert assistance via the ROCC device 8, and the communication pathway may, by way of nonlimiting illustrative example, comprise a telephonic or videoconference link established between the local operator LO and the assisting remote expert RE, along with sharing of the controller display and imaging bay sensor data with the remote expert RE. Advantageously, however, such involvement of the remote expert RE can be reduced or (in some cases) eliminated entirely by way of automated analysis of the remote monitoring 17, 18, 27 of the imaging examination acquired by the ROCC, thus enabling the ROCC to provide such assistance in an automated fashion without involvement of the remote expert RE thereby freeing the remote expert RE to handle more complex assistive tasks that cannot be performed in such automated fashion.

As disclosed herein, however, the ROCC framework is also used in the method 100 to provide assistive communication between two (or more) local operators. Since there may be dozens, hundreds, or more local operators performing imaging examinations under ROCC monitoring at any given time, the method 100 promotes targeted local operator-local operator assistance by identifying local operators that are likely to be in a position to assist one another.

### EXAMPLES

The foregoing are merely nonlimiting illustrative examples. The disclosed ROCC apparatus 1 is configured to output status or warning messages based on extracted images from the received examination monitoring information 17, 18, 27. Such messages can include, for example, missing or bad quality ECG trigger signals in cardiac examinations, low SNR coil signals, in-exam spurious test results (i.e., a leak in an RF cage or a not-properly closed door), low battery states of accessories, inappropriate selected motion compensation/fat suppression strategy for given patient signals, conflicts in scan parameters, high specific absorption rate (SAR), Peripheral Nerve Simulation (PNS), high dose values, exam dose level, tube temperature, and so forth.

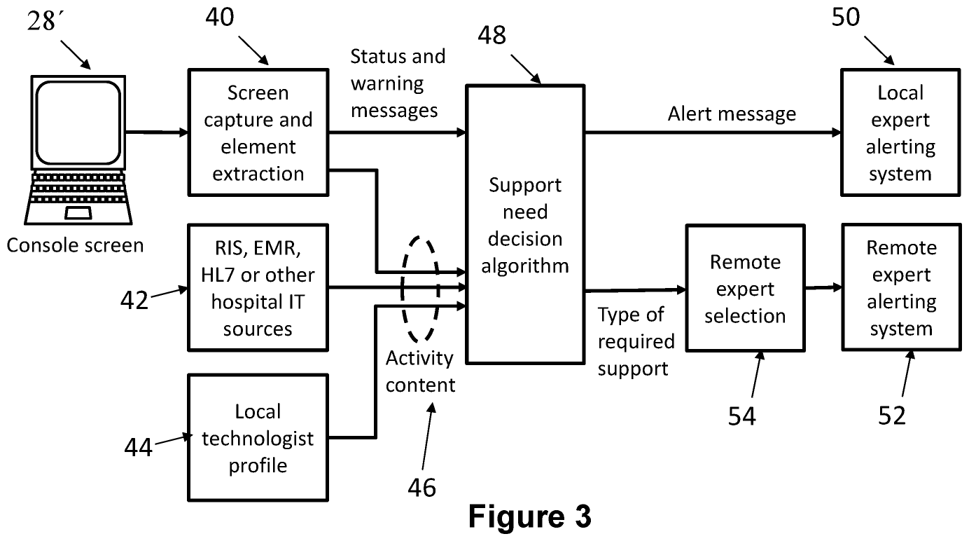

With reference to FIG. 3, and with continuing reference to FIGS. 1 and 2, the server computer 14s includes a screen capture module 40 configured to extract both status/warning messages and exam context from the received examination monitoring information 17, 18, 27. The exam context can include, but is not limited to, patient reference, type of exam/protocol, elapsed time, performed and planned sequences, time left in exam, and so forth.

The exam context is combined with detailed information about the patient and the imaging request from hospital IT systems 42 and with information about the local technologist profile 44 (i.e., areas of expertise) to form an activity context 46. The hospital IT systems 42 can include, for example, a radiology information system (RIS), an electronic medical record (EMR), or HL7 messaging. The video streams 17 of the in-room examination, showing the patient and/or the staff during exam preparation or during exam execution, can also be used as input, for example, to detect if a patient is already in the room, if the staff is still in the room, if the exam has started, etc.

The server computer 14s also includes a support need decision algorithm 48 configured to receive the status/warning messages and the combined activity context 46. The status/warning messages are interpreted in the activity context. For example, if a warning message states that the cardiac trigger signal is not received correctly and from the activity context it is known that a cardiac exam is running and data had already been acquired, it can be assumed that the local operator LO does not recognize the problem or does not know how to solve it. In this case, the decision algorithm 48 can decide to inform the local operator LO about potential image quality issues and the remote expert RE about support requirement for solving the trigger signal problem.

A local operator alerting module 50 will inform the local operator LO about the detected issue and the potential support need. A remote expert alerting module 52 will inform the remote expert RE about the need to contact the local site and provide support. In some embodiments, a remote expert selection module 54 is configured to select the remote expert RE with the best experience for the current support request.

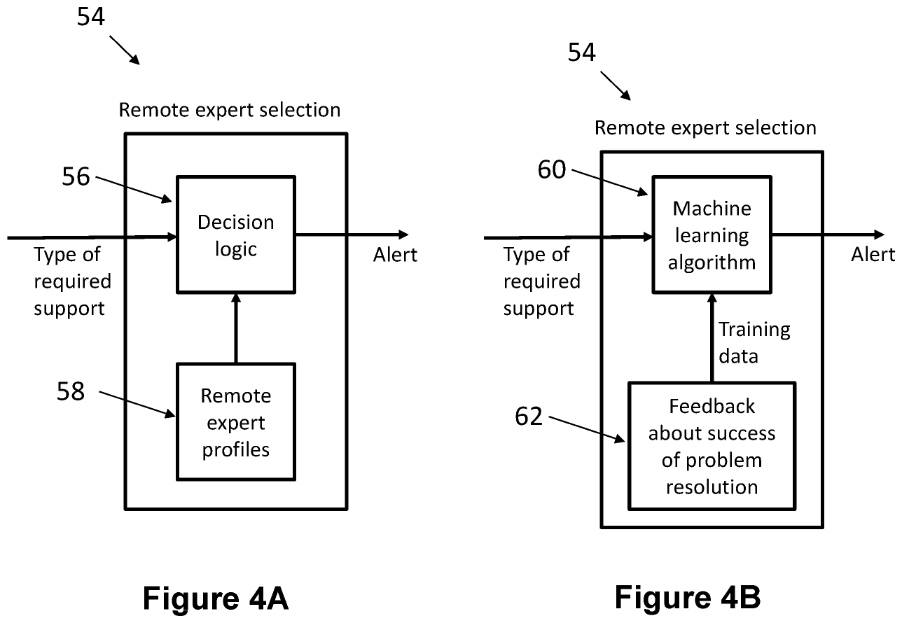

Examples of the remote expert selection module 54 are shown in FIGS. 4A and 4B. As shown in FIG. 4A, a decision logic module 56 can be supplied with a table of remote expert profiles 58 (i.e., specific expertise). In another example, as shown in FIG. 4B, a machine learning algorithm 60 can be employed and trained on a success measure obtained from local or remote expert feedback 62 about the success of the request or a combination thereof. For example, a remote expert profile may specify imaging device types (vendor, modality type, etc.) for which the remote expert RE can solve specific technical problems.

In some embodiments, the controller display GUI 28′ can display areas containing information to be extracted by the screen capture module 40. A first area on the controller display GUI 28′ can include exam context information, such as patient details, protocol type, and status and runtime of the individual sequences. A second area on the controller display GUI 28′ can include status and warning messages containing text to be extracted parsed, and interpreted. In some examples, the alert message for the local operator LO contains proposals or instructions for solving the problem without remote support. The instructions would be fetched from a database containing specific information for different types of delays. The instructions could be personalized or selected in a personalized way based on the local operator's expertise.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A non-transitory computer readable medium storing instructions for assisting an imaging examination of a patient, the instructions, when executed by a processor, cause the processor to:
obtain examination monitoring information related to the imaging examination and transmitting the examination monitoring information to a remote user the examination monitoring information including at least a screen mirroring data stream of a controller of an imaging device used to perform the imaging examination of the patient;
track a status of the imaging examination from the obtained examination monitoring information;
determine one or more indicators of an issue with the imaging examination based on the tracked status of the imaging examination; and
output a warning indicative of the determined one or more indicators of the issue with the imaging examination.

2. The non-transitory computer readable medium of claim 1, wherein to track the status, the instruction, when executed by the processor, causes the processor to one of:
perform an optical character recognition process on text from the examination monitoring information; and
match one or more templates with an image of the examination monitoring information.

3. The non-transitory computer readable medium of claim 1, wherein the examination monitoring information includes one or more of: a modality of the imaging device, an imaging scan being performed, and, a location of the patient relative to the imaging device.

4. The non-transitory computer readable medium of claim 1, wherein determining one or more indicators of an issue with the imaging examination based on the tracked status of the imaging examination includes:
detecting an error message displayed on a display of the controller of an imaging device the indicator of the issue being the error message and the issue being identified by the error message.

5. The non-transitory computer readable medium of claim 1, wherein determining one or more indicators of an issue with the imaging examination based on the tracked status of the imaging examination includes:
determining the issue as a deviation of the tracked status of the imaging examination from an expected imaging examination workflow that is expected for the imaging examination.

6. The non-transitory computer readable medium of claim 5, wherein the instructions, when executed by the processor, further cause the processor to:
obtain the expected workflow based on the obtained examination monitoring information

7. The non-transitory computer readable medium of claim 1, wherein the deviation of the tracked status of the imaging examination from the expected imaging examination workflow comprises an absence or a poor quality of an operative connection with the imaging device (2) of a component that is expected to be operatively connected based on an expected imaging examination workflow that is expected for the imaging examination.

8. The non-transitory computer readable medium of claim 7, wherein the component is an electrocardiogram or respiratory monitor and the operative connection with the ECG or respiratory monitor comprises a gating signal expected to be input to the imaging device by the ECG or respiratory monitor.

9. The non-transitory computer readable medium of claim 7, wherein the imaging device comprises a magnetic resonance imaging scanner and the component is an MRI coil, and the operative connection comprises a wired or wireless communication connection between the MRI coil and the MRI scanner.

10. The non-transitory computer readable medium of claim 1, wherein the imaging device comprises a magnetic resonance imaging (MRI) scanner and determining one or more indicators of an issue with the imaging examination based on the tracked status of the imaging examination includes:
determining the issue as radio frequency leakage of a Faraday cage enclosing the MRI scanner based on analysis of an MRI image acquired by the MRI scanner during the imaging examination.

11. The non-transitory computer readable medium of claim 1, wherein determining one or more indicators of an issue with the imaging examination based on the tracked status of the imaging examination includes:
identifying, from patient data, patient-specific considerations impacting the imaging examination.

12. The non-transitory computer readable medium of claim 1, wherein determining one or more indicators of an issue with the imaging examination based on the tracked status of the imaging examination includes:
identifying, from medical facility personnel data, a local operator profile for an operator performing the imaging examination;
wherein the issue is determined based on the tracked status of the imaging examination and further based on the local operator profile of the operator.

13. The non-transitory computer readable medium of claim 1, wherein the instruction, when executed by the processor, causes the processor to:
determine whether the one or more determined indicators of the issue with the imaging examination satisfies a predetermined warning threshold; and
output the warning when the one or more determined indicators of the issue exceed the predetermined warning threshold.

14. The non-transitory computer readable medium of claim 1, wherein the instruction, when executed by the processor, causes the processor to:
provide a natural communication pathway between a local operator performing the imaging examination and a remote user monitoring the medical examination.

15. The non-transitory computer readable medium of claim 1, wherein the instruction, when executed by the processor, causes the processor to:
display the warning on a display device.

16. The non-transitory computer readable medium of claim 15, wherein the display operable by a local operator performing the imaging examination or a remote user monitoring the imaging examination.

17. The non-transitory computer readable medium of claim 15, wherein the instruction, when executed by the processor, causes the processor to:
obtain, on the display device, an indication that the warning is resolved.

18. A system for assisting an imaging examination of a patient performed using an imaging device controlled by an imaging device controller, the system comprising:
a screen mirroring connection configured to perform screen mirroring of a display of the imaging device controller; and
a processor configured to:
obtain examination monitoring information related to the imaging examination and transmitting the examination monitoring information to a remote user the examination monitoring information including at least a screen mirroring data stream of the display of the imaging device controller acquired by the screen mirroring connection;
track a status of the imaging examination from the obtained examination monitoring information;
determine one or more indicators of an issue with the imaging examination based on the tracked status of the imaging examination; and
output, on a display, a warning indicative of the determined one or more indicators of the issue with the imaging examination.

19. The system of claim 18, further comprising:
an imaging device; and
an imaging device controller operatively connected to control the imaging device;
a camera arranged to acquire video of at least an area of the imaging device;
wherein the examination monitoring information further includes video acquired by the camera.

20. A method for assisting an imaging examination of a patient, the method comprising:
obtaining examination monitoring information related to the imaging examination and transmitting the examination monitoring information to a remote user the examination monitoring information including at least a screen mirroring data stream of a controller of an imaging device used to perform the imaging examination of the patient;
tracking a status of the imaging examination from the obtained examination monitoring information;
determining one or more indicators of an issue with the imaging examination based on the tracked status of the imaging examination; and
outputting a warning indicative of the determine one or more indicators of the issue with the imaging examination.
